(19) United States
(12) Patent Application Publication
(10) Pub. No.: US 2025/0073155 A1
(43) Pub. Date: Mar. 6, 2025

(54) HAIR CONDITIONER FOR STRAIGHTENING HAIR

(71) Applicant: HENKEL AG & CO. KGAA, Düsseldorf (DE)

(72) Inventors: Jens DELOWSKY, Norderstedt (DE); Sylvia KERL, Bönningstedt (DE)

(21) Appl. No.: 18/289,546

(22) PCT Filed: Jan. 9, 2023
(86) PCT No.: PCT/EP2023/050256

§ 371 Date: Nov. 3, 2023

(30) Foreign Application Priority Data
Mar. 23, 2022 (DE) 10 2022 202 842.2

(51) Int. Cl. A61K 8/92; A61Q 5/06

(52) U.S. Cl. CPC A61K 8/922; A61Q 5/06; A61K 2800/262; A61K 2800/31

(57) ABSTRACT

Anhydrous cosmetic compositions include specific amounts of cationic surfactants and ester oils in an ethanolic base, which are suitable for the non-chemical straightening or detangling of hair.

The present invention relates to a cosmetic composition for the non-chemical straightening or detangling of hair, which comprises cationic surfactants and ester oils in an ethanolic base, and which can be applied as a spray. The present invention further relates to a method for the non-chemical straightening or detangling of keratin fibers using the composition.

A lasting deformation of hair is usually carried out in such a way that the fibers are deformed mechanically and the deformation is fixed by suitable aids. Before and/or after this deformation, the fibers are treated with a keratin-reducing preparation. After a rinsing operation, the fibers are then treated with an oxidizing agent preparation in the so-called fixing step, rinsed, and freed of the deformation aids (rollers, papillotes) after or during the fixing step. If a mercaptan, for example ammonium thioglycolate, is used as the keratin-reducing component, this cleaves some of the disulfide bridges of the keratin molecule to —SH groups, resulting in softening of the hair fibers. During the subsequent oxidative fixing, disulfide bridges in the hair keratin are joined again so that the keratin structure is fixed in the predefined shape. Alternatively, instead of mercaptan, it is known to use sulfite for deforming hair. By means of hydrogen sulfite solutions and/or sulfite solutions and/or disulfite solutions, disulfide bridges of the keratin are cleaved in sulfitolysis according to the equation

R−S−S−R+HSO₃⁽⁻⁾ R−SH+R−S−SO₃⁽⁻⁾

and, in this way, the hair fibers are softened. Reducing compositions containing hydrogen sulfite, sulfite, or disulfite do not have the strong odor of the compositions containing mercaptan. The cleavage can be reversed, as outlined above, in a fixing step by means of an oxidizing agent to form new disulfide bridges.

The permanent straightening of keratin fibers can be achieved in an analogous manner by using keratin-reducing and keratin-oxidizing compositions. In such a method, the curly hair is either wound onto rollers having a large diameter of usually more than 15 mm or the hair is combed straight under the effect of the keratin-reducing composition. Instead of the roller, it is also possible to lay the fibers straight on a straightening board. Straightening boards are usually rectangular sheets, for example made of plastic. Preferably, the fiber is wetted with the keratin-reducing preparation during this process. A further possibility for straightening hair is straightening using hot irons.

The permanent straightening described above involves, as described, a chemical reduction and thus possibly permanent damage to the keratin fibers. In particular, if enhanced straightening is to be achieved, this is usually accompanied by increased damage to the keratin-containing fibers. This is not always desirable and non-chemical methods for straightening hair are therefore also known, wherein usually a cream-like emulsion containing care substances and conditioning substances is applied to the hair using a brush or similar and the hair is then straightened, for example using a straightening iron. This method is time-consuming and cumbersome since the cream must first be distributed evenly throughout the hair using a brush, and the straightening effect is not always satisfactory. It would therefore be desirable to have a non-chemical straightening composition which is easy to use and which has a long-lasting straightening effect, i.e., allows a greater number of hair washes without the straightening effect being lost.

DE 10216216312 proposes a multiphase, liquid composition as a non-chemical straightening composition, in which the aqueous phase predominantly contains water and the nourishing phase predominantly contains silicones.

However, water (and/or glycols in alternative formulations) is very polar and can cause increased curl formation, especially in wavy, curly, or frizzy hair. This can be particularly disadvantageous in dry hair if a water- (and/or glycol-)based straightening composition makes the blow-dried hair wavy or curly again. In addition, sustainability is of great importance when formulating new cosmetic compositions and efforts are being made to avoid the use of silicones as much as possible in their manufacture.

The object of the invention was therefore to provide a non-chemical straightening composition for hair fibers which has an excellent, sustainable straightening effect without the use of silicones and which is also easy to use.

Surprisingly, it has now been found that a non-chemical, silicone-free straightening composition having low viscosity and a high proportion of nourishing and/or conditioning substances is both easy to apply by spraying and has an excellent straightening effect for non-chemical straightening compositions.

The present invention is therefore characterized by the following statements:
1. A cosmetic composition for the non-chemical straightening or detangling of hair, containing
   a) 40-90 wt. % of ethanol,
   b) 0.1-5 wt. % of at least one cationic surfactant,
   c) 5-50 wt. % of at least one ester oil,
   wherein the composition contains less than 1 wt. % of water, and
   wherein all quantities stated relate to the total weight of the composition.
2. The composition according to statement 1, which has a viscosity in the range from 0 to 2000 mPas, preferably 100 to 800 mPas, more preferably 200 to 500 mPas (Brookfield RVDV-II+, 20° C., spindle 1, 100 rpm).
3. The composition according to any of the preceding statements, which is transparent.
4. The composition according to any of the preceding statements, wherein the total amount of ethanol ranges from 50-85 wt. % relative to the total weight of the composition.
5. The composition according to any of the preceding statements, wherein the total amount of ethanol ranges from 60-80 wt. % relative to the total weight of the composition.
6. The composition according to any of the preceding statements, wherein the total amount of the or each cationic surfactant b) ranges from 0.25-4 wt. % relative to the total weight of the composition.
7. The composition according to any of the preceding statements, wherein the total amount of the or each cationic surfactant b) ranges from 0.5-3 wt. % relative to the total weight of the composition.
8. The composition according to any of the preceding statements, wherein the or each cationic surfactant b) is selected from quaternary ammonium compounds.
9. The composition according to any of the preceding statements, wherein the or each cationic surfactant b) is selected from C₁-C₂₄ alkyltri(C₁-C₄)alkylammonium halide and/or methosulfate salts, preferably from cetyltrimethylammonium and behenyltrimethylammonium salts, and in particular from behenyltrimethylammonium salts, which contain a methosulfate and/or a chloride ion as anion.
10. The composition according to any of the preceding statements, wherein the total amount of the or each ester oil c) ranges from 10-45 wt. % relative to the total weight of the composition.
11. The composition according to any of the preceding statements, wherein the total amount of the or each ester oil c) ranges from 15-40 wt. % relative to the total weight of the composition.
12. The composition according to any of the preceding statements, wherein the or each ester oil c) is selected from carbonic acid esters, trifatty acid esters, esters of C6-C30 fatty acids with C2-C30 alcohols, or mixtures thereof.
13. The composition according to any of the preceding statements, wherein the or each ester oil c) is selected from symmetrical, asymmetrical, or cyclic esters of carbonic acid with C6-C12 fatty alcohols.
14. The composition according to any of the preceding statements, wherein the or each ester oil c) is selected from carbonic acid esters known under the INCI name dicaprylyl carbonate.
15. The composition according to statement 12, wherein the or each ester oil c) is selected from trifatty acid esters of saturated or unsaturated, linear or branched C6-C12 fatty acids and glycerol.
16. The composition according to statement 15, wherein the or each ester oil c) is selected from trifatty acid esters known under the INCI name caprylic/capric triglyceride.
17. The composition according to statement 12, wherein the or each ester oil c) is selected from esters of C8-C24 fatty acids with branched C2-C10 alcohols.
18. The composition according to statement 17, wherein the or each ester oil c) is selected from esters known under the INCI name isopropyl myristate.
19. The composition according to any of the preceding statements, further containing at least one vegetable oil in a weight proportion of 0.01-5 wt. %, based on the total weight of the composition.
20. The composition according to any of the preceding statements, which is packaged as a spray.
21. A cosmetic, non-medical method for the non-chemical straightening or detangling of hair, in which the composition according to any of the preceding statements is sprayed onto the hair and distributed throughout the hair, the hair is then dried and subsequently straightened mechanically and/or under the action of heat.
22. The method according to statement 21, comprising the following steps:
   a. washing the hair with a shampoo,
   b. applying the straightening composition to the hair by spraying without prior drying of the hair,
   c. straightening the hair mechanically and/or under the action of heat.
23. The method according to statement 22, wherein after step c., no more washing of the hair takes place.

The cosmetic compositions according to the invention are preferably liquid. According to the invention, the term “liquid” refers to the physical state at 1013 mbar and 20° C. “Liquid” means that the composition has such a viscosity that it can be easily applied by spraying. The composition preferably has a viscosity of 0 to 2000 mPas, more preferably 100 to 800 mPas and, in particular, 200 to 500 mPas (measured using Brookfield RVDV-II+, 20° C., spindle 1, 100 rpm).

The straightening composition is a solution which is preferably transparent to slightly turbid, and particularly preferably transparent.

In the context of the present invention, “transparency” means that the straightening composition preferably has an NTU (nephelometric turbidity unit) value of at most 100, preferably of at most 75, more preferably of at most 50, and in particular of at most 25 (measured, for example, with a Turbiquant® turbidimeter from Merck).

Since the presence of water can, in addition to the product-specific disadvantages as mentioned above, also cause optical disadvantages (for example turbidity due to insufficient solubilization of higher amounts of ester oils c) and/or perfume components), compositions according to the invention comprise less than 1 wt. % of water, based on the total weight of the composition. A maximum water content of <0.5 wt. % is even more preferred and the compositions are particularly preferably free of water.

According to the invention, “free of” means that no free water is added to the compositions. This does not include water as a by-product, which may be contained in other ingredients or commercial products.

For optimal product performance, it has proven to be preferable if ethanol is used in certain amounts, preferably in excess of the ester oil c).

The compositions according to the invention therefore particularly preferably comprise ethanol in a weight proportion of 50-85 wt. % and, in particular, 60-80 wt. % with respect to the total weight of the composition.

In order to achieve a hair-conditioning effect in addition to straightening or detangling of the hair fibers, the cosmetic compositions according to the invention contain 0.1-5 wt. % of at least one cationic surfactant b).

Suitable cationic surfactants for use in the compositions according to the invention are preferably cationic surfactants having a high hair care potential, a low foaming tendency, and the lowest possible emulsifying capacity.
Examples of cationic surfactants that can be used in the hair treatment compositions according to the invention include the following:
Linear or branched, saturated or unsaturated physiologically tolerated C₁-C₂₄ alkyltri(C₁-C₄)alkylammonium salts, di-C₁-C₂₄ alkyldi(C₁-C₄)alkylammonium salts or tri-C₁-C₂₄ alkyl(C₁-C₄)alkylammonium salts, for example halide and/or methosulfate salts, in particular chlorides, bromides and methosulfates, such as distearyldimethylammonium chloride, lauryltrimethylammonium chloride, lauryltrimethylbenzylammonium chloride, tricetylmethylammonium chloride, cetyltrimethylammonium chloride, cetyltrimethylammonium bromide, cetyltrimethylammonium methosulfate, stearyltrimethylammonium chloride, behenyltrimethylammonium chloride, behenyltrimethylammonium bromide and behenyltrimethylammonium methosulfate.
Very particular preference is given to C₁-C₂₄ alkyltri(C₁-C₄)alkylammonium halide and/or methosulfate salts and, in particular, cetyltrimethylammonium and behenyltrimethylammonium salts which contain a methosulfate and/or a chloride ion as anion.
Esterquats that contain both at least one ester function and at least one quaternary ammonium group as structural element. Preferred esterquats are quaternized ester salts of fatty acids with triethanolamine, quaternized ester salts of fatty acids with diethanolalkylamines and quaternized ester salts of fatty acids with 1,2-dihydroxypropyldialkylamines. Such products are marketed under the trademarks Stepantex®, Dehyquart®, and Armocare®, for example. The products Armocare® VGH-70, an N,N-bis(2-palmitoyloxyethyl)dimethylammonium chloride, and Dehyquart® F-75, Dehyquart® C-4046, Dehyquart® L80 and Dehyquart® AU-35 are examples of such esterquats.
Amidoamines, permanently cationic amidoamines and mixtures thereof.
Particularly suitable amidoamines can be selected from brassicamidopropyl dimethylamine, lauramidopropyl dimethylamine, myristamidopropyl dimethylamine, stearamidopropyl dimethylamine, cocamidopropyl dimethylamine, ricinolamidopropyl dimethylamine, isostearamidopropyl dimethylamine, oleamidopropyl dimethylamine, behenamidopropyl dimethylamine, palmamidopropyl dimethylamine, or mixtures thereof. Particular preference is given to brassicamidopropyl dimethylamine, behenamidopropyl dimethylamine, stearamidopropyl dimethylamine, or mixtures thereof.
Particularly suitable permanently cationic amidoamines can be selected from behenamidopropyl ethyldimonium ethosulfate, behenamidopropyl PG-dimonium chloride, oleamidopropyl ethyldimonium ethosulfate, oleamidopropyl PG-dimonium chloride, cocamidopropyl ethyldimonium ethosulfate, cocamidopropyl trimonium chloride, ricinoleamidopropyl ethyldimonium ethosulfate, rinoleamidopropyl trimonium chloride, ricinoleamidopropyl trimonium methosulfate, stearamidopropyl ethyldimonium ethosulfate, stearamidopropyl trimonium methosulfate, undecylamidopropyl trimonium methosulfate, lauramidopropyl PG-dimonium chloride, canolamidopropyl ethyldimonium ethosulfate, or mixtures thereof.

Quaternary ammonium compounds are preferably used as cationic surfactants b), since they can be incorporated particularly well into the alcoholic base of the compositions according to the invention. Particular preference is given to C₁-C₂₄ alkyltri(C₁-C₄)alkylammonium halide and/or methosulfate salts, very particular preference is given to cetyltrimethylammonium and behenyltrimethylammonium salts and, in particular, behenyltrimethylammonium salts which contain a methosulfate and/or a chloride ion as anion.

The cationic surfactant(s) b) is/are used in the compositions according to the invention preferably in a weight proportion of 0.25-4 wt. % and particularly preferably 0.5-3 wt. % with respect to the total weight of the composition.

As a third essential component, the compositions according to the invention contain 5-50 wt. % of at least one ester oil, based on the total weight of the compositions. It has been found that certain ester oils in the compositions according to the invention are excellent for hair straightening or detangling as well as for the simultaneous nourishing of the hair fibers and the scalp. They can completely replace silicones (or mineral oils) while maintaining the same effectiveness of corresponding compositions; treated hair appears smooth, glossy, and well-groomed and has a soft, non-greasy feel. Even after repeated use of the compositions according to the invention, there is no built-up effect to weigh down the hair.

In a preferred embodiment, the compositions according to the invention are therefore free of silicones and/or mineral oils.

Suitable ester oils according to the invention include, for example, the following:
i) Esters of C₆-C₃₀ fatty acids with C₂-C₃₀ alcohols. Preference is given to the monoesters of fatty acids with alcohols having 2 to 24 C atoms. Examples of fatty acid components used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, and technical mixtures thereof. Examples of the fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol and brassidyl alcohol and technical mixtures thereof. Particular preference according to the invention is given to isopropyl myristate (Rilanit® IPM), isononanoic acid C16-18 alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V). Isopropyl myristate is very particularly preferred.
ii) Dicarboxylic acid esters such as di-n-butyl adipate, di-(2-ethylhexyl) adipate, di-(2-ethylhexyl) succinate, and diisotridecyl acelaate and also diol esters such as ethylene glycol dioleate, ethylene glycol diisotridecanoate, propylene glycol di-(2-ethyl hexanoate), propylene glycol diisostearate, propylene glycol dipelargonate, butanediol diisostearate, neopentyl glycol dicaprylate.
iii) Symmetrical, asymmetrical, or cyclic esters of carbonic acid with fatty alcohols, preferably with C6-C12 fatty alcohols, for example glycerol carbonate or dicaprylyl carbonate. Very particular preference is given to symmetrical esters and, in particular, a carbonic acid ester known under the INCI name dicaprylyl carbonate, which is commercially available, for example, under the name Cetiol® CC, is preferred.
iv) Trifatty acid esters of saturated and/or unsaturated linear and/or branched fatty acids with glycerol, preferably C6-C12 fatty acids, and glycerol. Very particular preference is given to medium-chain glycerol triglycerides, for example trifatty acid esters known under the INCI name caprylic/capric triglyceride, which is available commercially, for example, under the name Myritol® 318.
v) Fatty acid partial glycerides, which are monoglycerides, diglycerides, and technical mixtures thereof. When technical products are used, small amounts of triglycerides may still be present due to the manufacturing process. The partial glycerides preferably follow the formula (D4-I),

CH₂O(CH₂CH₂O)ₘR¹

I

CHO(CH₂CH₂O)ₙR²

I

CH₂O(CH₂CH₂O)_{q}R³ (D4-I)

in which R¹, R² and R³, independently from one another, represent hydrogen or a linear or branched, saturated and/or unsaturated acyl group having 6 to 22, preferably 12 to 18, carbon atoms, with the proviso that at least one of these groups represents an acyl group and at least one of these groups represents hydrogen. The sum (m+n+q) represents 0 or numbers from 1 to 100, preferably 0 or 5 to 25. Preferably, R1 represents an acyl group and R2 and R3 represent hydrogen, and the sum (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, as well as the technical mixtures thereof. Oleic acid monoglycerides are preferably used.

The aforementioned ester oils can be used individually or in any desired mixtures in the compositions according to the invention.

Ester oils from the aforementioned groups i), iii) and iv) are preferred according to the invention.

In a preferred embodiment, compositions according to the invention contain symmetrical, asymmetrical, or cyclic esters of carbonic acid with C6-C12 fatty alcohols, preferably carbonic acid esters known under the INCI name dicaprylyl carbonate, as ester oil c).

In a further preferred embodiment, compositions according to the invention contain trifatty acid esters of saturated or unsaturated, linear or branched C6-C12 fatty acids and glycerol, preferably trifatty acid esters known under the INCI name caprylic/capric triglyceride, as ester oil c).

In a further preferred embodiment, compositions according to the invention contain esters of C8-C24 fatty acids with branched C2-C10 alcohols, preferably esters known under the INCI name isopropyl myristate, as ester oil c).

Ester oil(s) c) is/are used in the compositions according to the invention preferably in a weight proportion of 10-45 wt. % and particularly preferably 15-40 wt. % with respect to the total weight of the composition.

To further increase hair-nourishing properties, in particular to increase hair gloss, it can be advantageous if the compositions according to the invention further contain at least one vegetable oil in a weight proportion of 0.01-5 wt. % with respect to the total weight of the composition.

In the context of the present invention, suitable vegetable oils include, for example, amaranth seed oil, apricot kernel oil, argan oil, avocado oil, babassu oil, cottonseed oil, borage seed oil, camelina oil, safflower oil, peanut oil, pomegranate seed oil, grapefruit seed oil, hemp oil, rosehip seed oil, hazelnut oil, elderberry seed oil, currant seed oil, jojoba oil, cocoa butter, linseed oil, macadamia nut oil, corn germ oil, almond oil, marula oil, evening primrose oil, olive oil, palm oil, peach kernel oil, rapeseed oil, rice oil, sea buckthorn pulp oil, sea buckthorn seed oil, sesame oil, shea butter, soybean oil, sunflower oil, grape seed oil, walnut oil, and/or wild rose oil.

Particularly suitable are the above-mentioned vegetable oils that are very easy to distribute on the scalp and throughout the hair and are absorbed well and quickly into the hair fibers and nourish them, for example babassu oil.

The compositions according to the invention can also contain common components of cosmetic compositions, such as preservatives, perfumes, fragrances, or pH adjusters. Emulsifiers can also be contained in small amounts, or the components already mentioned can have an emulsifying effect in order to solubilize perfume or other components even better, as long as the overall visual appearance of the composition is not changed.

The straightening composition according to the invention is preferably applied to the hair by spraying. For this purpose, it is preferably packaged as a spray in an appropriate container.

The present invention also relates to a cosmetic, non-medical method for the non-chemical straightening or detangling of hair, in which the composition according to the invention is sprayed onto the hair and distributed throughout the hair, the hair is then dried and subsequently straightened mechanically and/or under the action of heat. The method according to the invention has the advantage of being particularly simple and time-saving, since shaking of the compositions (such as in the case of two-phase compositions) or the use of an aid such as a brush or similar is not required since, thanks to its low viscosity, the composition can be easily sprayed and penetrates the hair strands well.

The method for straightening hair using the straightening composition also ensures a significantly reduced overall time for straightening hair. More precisely, a preferred method for straightening keratin fibers according to the invention may comprise the following steps:
a. washing the hair with a shampoo,
b. applying the straightening composition to the hair by spraying without prior drying of the hair,
c. straightening the hair mechanically and/or under the action of heat.
   Preferably, after step c. (straightening the hair), no more washing of the hair takes place.
   The method for straightening keratin fibers is thereby significantly shortened compared to conventional straightening methods with non-chemical straightening compositions.

Conventional straightening methods for non-chemical straightening usually involve the following steps: washing the hair with a shampoo, drying the hair by blow-drying, applying the straightening composition using a brush strand by strand, drying the hair by blow-drying, straightening, washing again with a shampoo, and treating with a conditioner and blow-drying. In contrast, the straightening method according to the invention has fewer treatment steps and the straightening composition itself can also be applied more quickly thanks to the low viscosity and easy spraying. At the same time, a straightening effect was achieved which is comparable to that of the top products from well-known non-chemical straightening compositions. This relates both to the appearance of the hair immediately after straightening and to the duration of the straightening effect. With the composition according to the invention, for example, excellent straightening of the hair could be achieved, which lasted until the next hair wash without any significant loss of the straightening effect.

With regard to preferred embodiments of the method, the same applies, mutatis mutandis, as to the liquid straightening composition.

### EXAMPLES

The composition of some preferred liquid straightening compositions can be found in the following tables (amounts given in wt. % based on the total weight of the composition, unless otherwise indicated).

| | Formula 1 | Formula 2 | Formula 3 |
|---|---|---|---|
| a) Ethanol | 40-90 | 50-85 | 60-80 |
| b) Cationic surfactant | 0.1-5 | 0.25-4 | 0.5-3 |
| c) Ester oil | 5-50 | 10-45 | 15-40 |
| Misc. | up to 100 | up to 100 | up to 100 |

| | Formula 4 | Formula 5 | Formula 6 |
|---|---|---|---|
| a) Ethanol | 40-90 | 50-85 | 60-80 |
| b) Quaternary ammonium compound | 0.1-5 | 0.25-4 | 0.5-3 |
| c) Carbonic acid esters, | 5-50 | 10-45 | 15-40 |
| trifatty acid esters, | | | |
| esters of C6-C30 fatty | | | |
| acids with C2-C30 | | | |
| alcohols, or mixtures thereof | | | |
| Misc. | up to 100 | up to 100 | up to 100 |

| | Formula 7 | Formula 8 | Formula 9 |
|---|---|---|---|
| a) Ethanol | 40-90 | 50-85 | 60-80 |
| b) Quaternary | 0.1-5 | 0.25-4 | 0.5-3 |
| ammonium compound | | | |
| c) Carbonic acid esters, | 5-50 | 10-45 | 15-40 |
| trifatty acid esters, esters | | | |
| of C6-C30 fatty acids | | | |
| with C2-C30 alcohols, or | | | |
| mixtures thereof | | | |
| d) Vegetable oil | 0-5 | 0.01-5 | 0.025-3 |
| Misc. | up to 100 | up to 100 | up to 100 |

| | Formula 10 | Formula 11 | Formula 12 |
|---|---|---|---|
| a) Ethanol | 40-90 | 50-85 | 60-80 |
| b) INCI: cetrimonium chloride | 0.1-5 | 0.25-4 | 0.5-3 |
| and/or INCI: | | | |
| behentrimonium chloride | | | |
| and/or INCI: | | | |
| cetrimonium methosulfate | | | |
| and/or INCI: | | | |
| behentrimonium methosulfate | | | |
| c) INCI: dicaprylyl carbonate | 5-50 | 10-45 | 15-40 |
| d) Vegetable oil | 0-5 | 0.01-5 | 0.025-3 |
| Misc. | up to 100 | up to 100 | up to 100 |

| | Formula 13 | Formula 14 | Formula 15 |
|---|---|---|---|
| a) Ethanol | 40-90 | 50-85 | 60-80 |
| b) INCI: cetrimonium chloride | 0.1-5 | 0.25-4 | 0.5-3 |
| and/or INCI: | | | |
| behentrimonium chloride | | | |
| and/or INCI: | | | |
| cetrimonium methosulfate | | | |
| and/or INCI: | | | |
| behentrimonium methosulfate | | | |
| c) INCI: isopropyl myristate | 5-50 | 10-45 | 15-40 |
| d) Vegetable oil | 0-5 | 0.01-5 | 0.025-3 |
| Misc. | up to 100 | up to 100 | up to 100 |

| | Formula 16 | Formula 17 | Formula 18 |
|---|---|---|---|
| a) Ethanol | 40-90 | 50-85 | 60-80 |
| b) INCI: cetrimonium chloride | 0.1-5 | 0.25-4 | 0.5-3 |
| and/or INCI: | | | |
| behentrimonium chloride | | | |
| and/or INCI: | | | |
| cetrimonium methosulfate | | | |
| and/or INCI: | | | |
| behentrimonium methosulfate | | | |
| c) INCI: caprylic/capric | 5-50 | 10-45 | 15-40 |
| triglyceride | | | |
| d) Vegetable oil | 0-5 | 0.01-5 | 0.025-3 |
| Misc. | up to 100 | up to 100 | up to 100 |

“Misc.” refers to other common ingredients, such as preservatives, fragrances, perfumes, pH regulators.

1. A cosmetic composition for non-chemical straightening or detangling of hair, the composition comprising:
40-90 wt. % of ethanol;
0.1-5 wt. % of at least one cationic surfactant;
5-50 wt. % of at least one ester oil; and
less than 1 wt. % of water.

2. The composition of claim 1, wherein the composition has a viscosity in the range from 0 to 2000 mPas.

3. The composition of claim 1, wherein the composition is transparent.

4. The composition of claim 1, wherein the at least one cationic surfactant comprises a quaternary ammonium compound.

5. The composition of claim 1, wherein the at least one cationic surfactant comprises a C1-C24 alkyltri(C1-C4)alkylammonium halide and/or methosulfate salt.

6. The composition of claim 1, wherein the at least one ester oil comprises a carbonic acid ester, a trifatty acid ester, an ester of C6-C30 fatty acids with C2-C30 alcohols, or mixtures thereof.

7. The composition of claim 1, wherein the at least one ester oil comprises a symmetrical, an asymmetrical, or a cyclic ester of carbonic acid with C6-C12 fatty alcohols.

8. The composition of claim 1, wherein the at least one ester oil comprises a trifatty acid ester of saturated or unsaturated, linear or branched C6-C12 fatty acid and glycerol.

9. The composition of claim 1, wherein the at least one ester oil comprises an ester of C8-C24 fatty acids with branched C2-C10 alcohols.

10. The composition of claim 1, further comprising at least one vegetable oil in a weight proportion of 0.01-5 wt. %.

11. A spray comprising the composition of claim 1.

12. A cosmetic, non-medical method for non-chemical straightening or detangling of hair, the method comprising:
spraying the composition of claim 1 onto the hair;
distributing the composition throughout the hair;
drying the hair; and
mechanically straightening and/or heating the hair.

13. The method of claim 12, further comprising:
washing the hair with a shampoo before spraying the composition onto the hair,
wherein spraying the composition onto the hair is done without prior drying of the hair.

14. The method of claim 13, wherein further washing of the hair is not done after mechanically straightening and/or heating the hair.

15. The composition of claim 2, wherein the viscosity of the composition is in the range from 100 to 800 mPas.

16. The composition of claim 15, wherein the viscosity of the composition is in the range from 200 to 500 mPas.

17. The composition of claim 5, wherein the C1-C24 alkyltri(C1-C4)alkylammonium halide and/or methosulfate salt is cetyltrimethylammonium and/or behenyltrimethylammonium salt.

18. The composition of claim 17, wherein the cetyltrimethylammonium and/or behenyltrimethylammonium salt is behenyltrimethylammonium salt having a methosulfate and/or a chloride ion as an anion.

19. The composition of claim 7, wherein the at least one ester oil comprises dicaprylyl carbonate.

20. The composition of claim 8, wherein the at least one ester oil comprises caprylic/capric triglyceride.
